(19) United States
(12) Patent Application Publication
(10) Pub. No.: US 2004/0203048 A1
(43) Pub. Date: Oct. 14, 2004

(54) High-throughput DNA methylation profiling and comparative analysis

(76) Inventor: Nathaniel Tue Tran, Irvin, CA (US)

Correspondence Address: Nathaniel Tue Tran, 3205 Aspen Grove, Irvine, CA, 92618, US

(21) Appl. No.: 10/835,028

(22) Filed: Apr. 28, 2004

Related U.S. Application Data
(63) Continuation-in-part of application No. 10/680,277, filed on Oct. 7, 2003.
(63) Continuation-in-part of application No. 10/667,882, filed on Sep. 22, 2003.
(60) Provisional application No. 60/443,017, filed on Jan. 28, 2003.

(51) Int. Cl. C12Q 1/68; C12P 19/34

(52) U.S. Cl. 435/6; 435/91.2

(57) ABSTRACT

This invention teaches high-throughput process for quantifying and comparing DNA methylation in multiple genes. DNA is extracted and subject to treatments that differentially modify either non-methylated nucleotides or methylated nucleotides. The modification results in labels or detectable tags that can easily be detected and quantified. The treated DNA is then digested by restriction enzymes and profiled on DNA array. The method can be used to compare two samples of DNA to look for differentially methylated genes. The method can also reveal polymorphism besides epigenetic differences.

### CROSS-REFERENCE TO RELATED APPLICATIONS

This application is a continuation in-part of U.S. patent application Ser. No. 10/680,277 filed Oct. 7, 2003 and U.S. Ser. No. 10/667,882 filed Sep. 22, 2003. This application also incorporates another invention described in U.S. provisional patent application serial No. 60/559,685 filed Apr. 2, 2004.

### TECHNICAL FIELD OF THE INVENTION

The present invention provides a method for quickly profiling, identifying, quantifying and comparing methylation of DNA in many genes simultaneously. The method combines the use of DNA differential labeling by methylation events and DNA array.

### BACKGROUND OF THE INVENTION

The degree of DNA methylation changes in many forms of cancers. When DNA of a gene is heavily methylated, that particular gene is no longer actively transcribed and translated into functional proteins. When such a gene keeps cell division in check, the resulting hypermethylation of this gene can lead to cancer conditions. A method for identifying methylation or change in pattern of methylation can be used to identify methylation markers that are indicative of cancers or onset of cancers and then used to diagnose those conditions.

In higher order eukaryotic organisms, DNA is frequently methylated at cytosines located 5′ to guanosines in the CpG dinucleotides. This modification has important regulatory effects on gene expression predominantly when it involves CpG rich areas (CpG islands) located in the promoter region of a gene sequence. Extensive methylation of CpG islands has been associated with transcriptional inactivation of selected imprinted genes and genes on the inactive X chromosome of females. Aberrant methylation of normally unmethylated CpG islands has been described as a frequent event in immortalized and transformed cells and has been frequently associated with transcriptional inactivation of tumor suppressor genes in human cancers.

DNA is often methylated in normal mammalian cells. For example, DNA is methylated to determine whether a given gene will be expressed and whether the maternal or the paternal allele of that gene will be expressed; See Melissa Little et al., Methylation and p16: Suppressing the Suppressor, 1 NATURE MEDICINE 633 (1995). While methylation is known to occur at CpG sequences, only recent studies indicate that CpNpG sequences may be methylated. Susan J. Clark et al., CpNpGp Methylation in Mammalian Cells, 10 NATURE GENETICS 20, 20 (1995). Methylation at CpG sites has been much more widely studied and is better understood.

Methylation occurs by enzymatic recognition of CpG and CpNpG sequences followed by placement of a methyl (CH₃) group on the fifth carbon atom of a cytosine base. The enzyme that mediates methylation of CpG dinucleotides, 5-cytosine methyltransferase, is essential for embryonic development—without it embryos die soon after gastrulation. It is not yet clear whether this enzyme methylates CpNpG sites. Peter W. Laird et al., DNA Methylation and Cancer, 3 HUMAN MOLECULAR GENETICS 1487, 1488 (1994).

DNA methylases transfer methyl groups from a universal methyl donor, such as S- Adenosyl-L-Methionine (SAM), to specific sites on the DNA. One biological function of DNA methylation in bacteria is protection of the DNA from digestion by cognate restriction enzymes. Mammalian cells possess methylases that methylate cytosine residues on DNA that are 5′ neighbors of guanine residues (CpG). This methylation may play a role in gene inactivation, cell differentiation, tumorigenesis, X-chromosome inactivation, and genomic imprinting. CpG islands remain unmethylated in normal cells, except during X-chromosome inactivation and parental specific imprinting where methylation of 5′ regulatory regions can lead to transcriptional repression. DNA methylation is also a mechanism for changing the base sequence of DNA without altering its coding function. DNA methylation is a heritable, reversible epigenetic change. Yet, DNA methylation has the potential to alter gene expression, which has profound developmental and genetic consequences.

The methylation reaction involves flipping a target cytosine out of an intact double helix to allow the transfer of a methyl group from S-adenosyl-methionine in a cleft of the enzyme DNA (cystosine-5)-methyltransferase to form 5-methylcytosine (5-mCyt). This enzymatic conversion is the only epigenetic modification of DNA known to exist in vertebrates and is essential for normal embryonic development. The presence of 5-mCyt at CpG dinucleotides has resulted in a 5-fold depletion of this sequence in the genome during vertebrate evolution, presumably due to spontaneous deamination of 5-mCyt to T (Schoreret et al., Proc. Natl. Acad Sci. USA 89:957-961, 1992). Those areas of the genome that do not show such suppression are referred to as “CpG islands” (Bird, Nature 321:209-213, 1986; and Gardiner-Garden et al., J. Mol. Biol. 196:261-282, 1987). These CpG island regions comprise about 1% of vertebrate genomes and also account for about 15% of the total number of CpG dinucleotides (Bird, Nature 321:209-213, 1986). CpG islands are typically between 0.2 to about 1 kb in length and are located upstream of many housekeeping and tissue-specific genes, but may also extend into gene coding regions. Therefore, it is the methylation of cytosine residues within CpG islands in somatic tissues, which is believed to affect gene function by altering transcription (Cedar, Cell 53:3-4, 1988).

When a gene has many methylated cytosines it is less likely to be expressed. Hence, if a maternally-inherited gene is more highly methylated than the paternally-inherited gene, the paternally-inherited gene will give rise to more gene products. Similarly, when a gene is expressed in a tissue-specific manner, that gene will often be unmethylated in the tissues where it is active, but will be highly methylated in the tissues where it is inactive. Incorrect methylation is thought to be the cause of some diseases including Beckwith-Wiedemann syndrome and Prader-Willi syndrome. I. Henry et al., 351 NATURE 665, 667 (1991); R. D. Nicholls et al., 342 NATURE 281, 281-85 (1989).

The degree of methylation of cytosine residues contained within CpG islands of certain genes has been inversely correlated with gene activity. This could lead to decreased gene expression by a variety of mechanisms including, for example, disruption of local chromatin structure, inhibition of transcription factor-DNA binding, or by recruitment of proteins which interact specifically with methylated sequences indirectly preventing transcription factor binding. In other words, there are several theories as to how methylation affects mRNA transcription and gene expression, but the exact mechanism of action is not well understood. Some studies have demonstrated an inverse correlation between methylation of CpG islands and gene expression, however, most CpG islands on autosomal genes remain unmethylated in the germline and methylation of these islands is usually independent of gene expression. Tissue-specific genes are usually unmethylated in the receptive target organs but are methylated in the germline and in non-expressing adult tissues. CpG islands of constitutively-expressed housekeeping genes are normally unmethylated in the germline and in somatic tissues.

Abnormal methylation of CpG islands associated with tumor suppressor genes may also decrease their expression. Increased methylation of such regions may lead to progressive reduction of normal gene expression resulting in the selection of a population of cells having a selective growth advantage (i.e., a malignancy).

It is considered that an altered DNA methylation pattern, particularly methylation of cytosine residues, causes genome instability and mutagenesis. This, presumably, has led to an 80% suppression of a CpG methyl acceptor site in eukaryotic organisms, which methylate their genomes. Cytosine methylation further contributes to generation of polymorphism and germ-line mutations and to transition mutations that inactivate tumor-suppressor genes (Jones, Cancer Res. 56:2463-2467, 1996). Methylation is also required for embryonic development of mammals (Li et al., Cell 69:915-926, 1992). It appears that the methylation of CpG-rich promoter regions may be blocking transcriptional activity. Ushijima et al. (Proc. Natl. Acad Sci. USA 94:2284-2289, 1997) characterized and cloned DNA fragments that show methylation changes during murine hepato-carcinogenesis. Data from a group of studies of altered methylation sites in cancer cells show that it is not simply the overall levels of DNA methylation that are altered in cancer, but changes in the distribution of methyl groups.

Most molecular biological techniques used to analyze specific loci, such as CpG islands in complex genomic DNA, involve some form of sequence-specific amplification, whether it is biological amplification by cloning in E. coli, direct amplification by PCR or signal amplification by hybridization with a probe that can be visualized. Since DNA methylation is added post-replication by a dedicated maintenance DNA methyl-transferase that is not present in either E. coli or in the PCR reaction, such methylation information is lost during molecular cloning or PCR amplification. Moreover, molecular hybridization does not discriminate between methylated and none-methylated DNA, since the methyl group on the cytosine does not participate in base pairing. The lack of a facile way to amplify the methylation information in complex genomic DNA has probably been a most important impediment to DNA methylation research. Therefore, there is a need in the art to improve upon methylation detection techniques, especially in a quantitative manner.

The indirect methods for DNA methylation pattern determinations at specific loci that have been developed rely on techniques that alter the genomic DNA in a methylation-dependent manner before the amplification event. There are two primary methods that have been utilized to achieve this methylation-dependent DNA alteration. The first is digestion by a restriction enzyme that is affected in its activity by 5-methylcytosine in a CpG sequence context. The cleavage, or lack of it, can subsequently be revealed by Southern blotting or by PCR. The other technique that has received recent widespread use is the treatment of genomic DNA with sodium bisulfite. Sodium bisulfite treatment converts all unmethylated cytosines in the DNA to uracil by deamination, but leaves the methylated cytosine residues intact. Subsequent PCR amplification replaces the uracil residues with thymines and the 5-methylcytosine residues with cytosines. The resulting sequence difference has been detected using standard DNA sequence detection techniques, primarily PCR.

Many DNA methylation detection techniques utilize bisulfite treatment. Currently, all bisulfite treatment-based methods are followed by a PCR reaction to analyze specific loci within the genome. There are two principally different ways in which the sequence difference generated by the sodium bisulfite treatment can be revealed. The first is to design PCR primers that uniquely anneal with either methylated or unmethylated converted DNA. This technique is referred to as “methylation specific PCR” or “MSP”. The method used by all other bisulfite-based techniques (such as bisulfite genomic sequencing, COBRA and Ms-SNuPE) is to amplify the bisulfite-converted DNA using primers that anneal at locations that lack CpG dinucleotides in the original genomic sequence. In this way, the PCR primers can amplify the sequence in between the two primers, regardless of the DNA methylation status of that sequence in the original genomic DNA. This will result in a pool of different PCR products, all with the same length and differing in their sequence only at the sites of potential DNA methylation at CpGs located in between the two primers. The difference between these methods of processing the bisulfite-converted sequence is that in MSP, the methylation information is derived from the occurrence or lack of occurrence of a PCR product, whereas in the other techniques a mix of products is always generated and the mixture is subsequently analyzed to yield quantitative information on the relative occurrence of the different methylation states.

MSP is mostly a qualitative technique. There are two reasons that it is not quantitative. The first is that methylation information is derived from the comparison of two separate PCR reactions (the methylated and the non-methylated version). There are inherent difficulties in making kinetic comparisons of two different PCR reactions. The other problem with MSP is that often the primers cover more than one CpG dinucleotide. The consequence is that multiple sequence variants can be generated, depending on the DNA methylation pattern in the original genomic DNA. For instance, if the forward primer is a 24-mer oligonucleotide that covers 3 CpGs, then 2\hat{ }3=8 different theoretical sequence permutations could arise in the genomic DNA following bisulfite conversion within this 24-nucleotide sequence. If only a fully methylated and a fully unmethylated reaction is run, then you are really only investigating 2 out of the 8 possible methylation states. The situation is further complicated if the intermediate methylation states lead to amplification, but with reduced efficiency.

Regardless of which techniques used, the limitation is to just one specific gene or one specific locus in the genome can be targeted and analyzed at a time. Such techniques would be heavily biased toward known methylation sites, well-known genes instead of a general exploration of any genes that may got methylated. As a result, these techniques are not well suitable for biomarker discovery. Therefore there is still a need for a method that can profile and quantify or compare the degree of methylation of many genes simultaneously.

### SUMMARY OF THE INVENTION

The present invention provides a method for measuring and comparing DNA methylation in many genes simultaneously. The method comprises the steps of:

(1) treating a DNA sample with a modifying agent that differentially modifies non-methylated vs. methylated nucleotides with labeled donor groups;

(2) subjecting DNA to restriction digest;

(3) profiling DNA fragments on a DNA array; and

(4) quantifying labels for comparing between arrays to identify aberrant methylation pattern.

DNA can be differentially modified by methylation using labeled methyl donors containing ³H or ¹⁴C. Non-methylated DNA will take up labels while methylated DNA won't. Two DNA samples can be separately treated with ³H for one and ¹⁴C for the other and then combined for restriction digestion and profiling on the same DNA array. Radiation signals from ³H can be separately quantified from signals from ¹⁴C using existing technologies known to those skilled in the art. These signals are then compared to identify any methylation variation between the two samples.

Alternatively both DNA samples can be labeled with the same label and treated separately and then profiled on two identical arrays for analysis. As long as there are sufficient amount of DNA in both samples to substantially saturate all the aptamers binding capacity in every spot, then quantitative comparison is relatively easy and straight forward. This method of analysis also allows longitudinal studies where new DNA is assayed and compared to historical DNA assay results stored in a database. Methylation pattern of DNA from a normal population can be established and then used to find meaningful aberration in methylation pattern of DNA from a sick population. Sometimes the differences yielded are due to polymorphism (DNA sequence difference) causing differential restriction digest. The discovery of such polymorphism is also of interest for biomarker discovery purpose.

One object of the invention is to allow an investigator to quickly identify where the methylation varies in various genes from one source of DNA to another. This is accomplished by capturing different genes using complementary sequences of DNA on DNA arrays, and quantifying the degree of methylation by counting the labels added onto the DNA.

Another object of the invention is to allow rapid detection of aberration in DNA methylation. Such detection can be used to detect cancer or early onset of cancer condition. With this method, the DNA methylation pattern can also be used to follow the course of cancer.

A further object of the invention is to identify the genes with differential methylation. Such genes are implicated in the cause of the diseased condition or can yield potential therapeutic targets. The genes discovered can also be used to better understand the disease mechanism and thus better design a diagnostic or therapeutic approach.

A further object of the invention is to use the DNA methylation pattern as a tool for sub-typing cancers or other diseased conditions. Such sub-typing classification can lead to better therapeutic targeting. Additionally, it can also lead to better prediction and prognosis of the disease progression.

A further object of the invention is to use this method for biomarker discovery. Signal differences between arrays can be traced to differential methylation of certain genes or gene polymorphism resulting in differential cleavage by restriction enzymes. Either way, if any difference discovered is responsible for a diseased condition or other things; then such difference can be exploited to devise a method to diagnose the disease or develop a therapeutic to treat the disease.

### BRIEF DESCRIPTION OF THE DRAWINGS

FIG. 1 describes the process of comparing DNA methylation from two DNA samples using the same label on two identical DNA arrays.

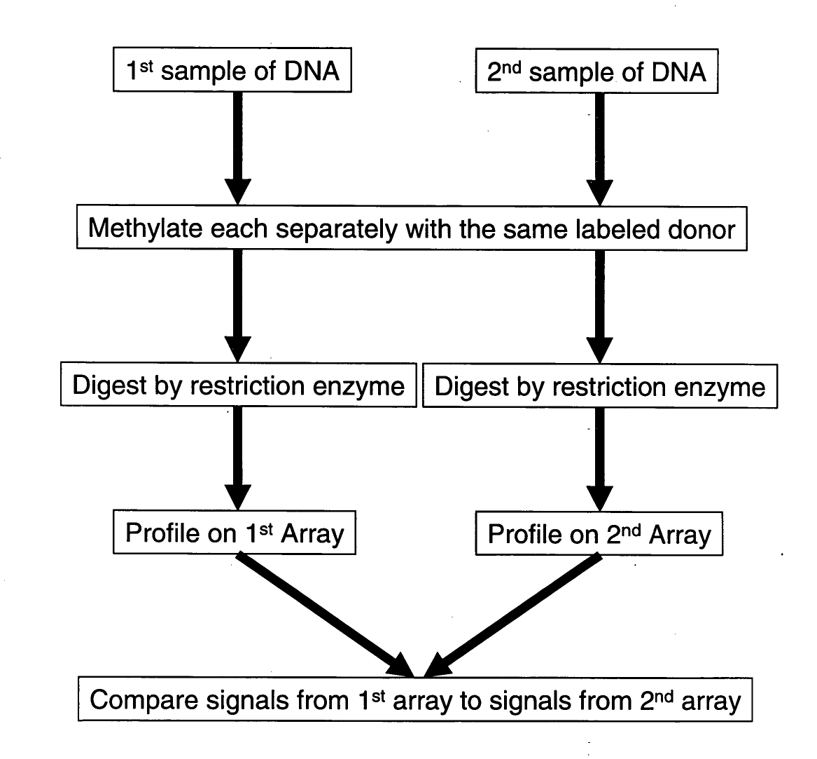

FIG. 2 describes the process of comparing DNA methylation from two DNA samples using different isotopes on a single DNA array.

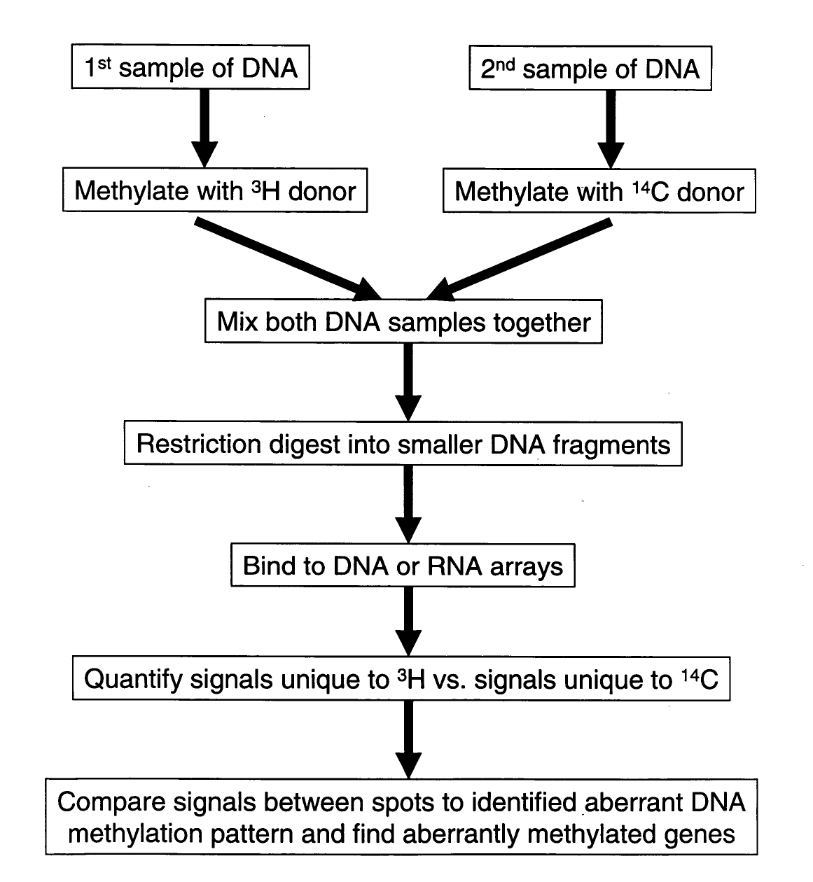

### DETAIL DESCRIPTION OF THE INVENTION

The principle of the invention is when DNA has received methylation at a particular site, then that site cannot receive any more of the same methylation or similar types of modifications. Thus by taking DNA and methylating them externally using labeled methyl groups, one can quantify labels and gain insights into preexisting methylation. More specifically, one can use this method to compare the degree of preexisting methylation between two DNA samples. For instance, these DNA samples are digested into fragments and profiled on DNA arrays so that specific genes are captured at specific locations, then the signal read at these locations can be used to compare the degree of specific gene methylation.

Methods for Comparing DNA Methylation

Take two DNA samples that are sufficiently purified and quantified to be equivalent for comparison. Then subject these samples to methylation with labeled donor methyls such as the commercially available Adenosyl-L-Methionine, S-[methyl-¹⁴C]; Adenosyl-L-Methionine, S-[methyl-³H], and an enzyme such as mammalian DNA methyltransferase AKA DNA methylase. The methylation reaction will selectively label certain non-methylated sites thus DNA with fewer preexisting methylation will get more labels and vice-versa. After labeling, DNA is cleaned up and subjected to restriction digestion to cut DNA into smaller and more manageable fragments. These fragments are then profiled on DNA array which will capture complementary sequences and display them at specific location for quantitative comparison. If the identical arrays are used for both DNA samples then their results will be comparable. The comparison is further made more reliable by having sufficient amount of DNA in each sample to saturate all the binding capacity of every spot on these arrays. When such saturation is achieved, not only relative amount of methylation between genes can be compared, but absolute amount can also be compared for every particular gene.

Using the above procedure, the DNA methylation pattern from one patient's sample can be profiled and then stored for comparison to samples of the same patient or those of other patients taken later on in time. Using this setup, DNA methylation patterns of cancer patients can be monitored over time for indication or correlation with their diseased condition. Using a standard and established procedure such as one outlined in a methylation labeling kit and a standard DNA array, one can also study a patient DNA methylation pattern and compared it to those of others stored in a data bank. It is also possible to use DNA methylation pattern to diagnose and subtype a cancer once a correlation is established. Such diagnosis and subtyping enable better therapeutic targeting.

Another improved way to compare DNA methylation in two DNA samples is to label one with ³H-label and label the other with ¹⁴C-label. Then combine the two samples into a mixture for restriction digestion and profiling on the same DNA array. Radiation signal resulting from ³H can be quantitatively separated from radiation signal resulting from ¹⁴C. There are different ways to separately quantify the two radiation signal: one is to use a selective blocking screen between the radiation source and the detector, and the other is to isolate the different radiation signals detected based on characteristic such as pulse height in scintillation counting. Radiation from ³H is relatively weaker than radiation from ¹⁴C thus a blocking screen will selectively block more ³H radiation than 14C radiation. As a result, quantifying radiation with and without a blocking screen can be used to calculate radiation specific to each isotope. The pulse height method is possible with scintillation counting because radiation from ¹⁴C can strike more scintillation materials thus generating higher pulse of photons. This method has been well-known to those skilled in the art and has been used to enable scintillation counter to quantify a specific isotope in a mixture. Some of these methods are described in U.S. Pat. Nos. 4,628,205; 4,918,310; 5,134,294; 5,753,917.

There are many ways to perform methylation reactions. The enzymatic ways is most preferred using enzymes such as DNA methylase and methyl donors such as S-Adenosyl-Methionine. Some of the DNA methylases may not modify all DNA bases, but only cytosine in CpGs, or CpNpG as recently discovered. Such limitation makes these enzymes even better suited for this type of study. Other methods or methylation are available such as chemical methylation using a methylating agent that will directly modified DNA such as dimethyl sulfate. These methods while not discussed here in detail are commonly known to those skilled in the art and can be used as alternatives for performing this experiment. Dimethyl sulfate with radioactive methyl groups can easily be used to methylate all remaining DNA bases that are not yet methylated and then subject the resulting DNA to restriction digestion and array analysis.

While adding a methyl group is the preferred method of choice because one does not have to worry about differential digestion by restriction enzymes, other forms of modifications can also be used. Other Alkylating agents that add bigger groups such as ethyl, propyl, isopropyl . . . or even a group linked to a biotin tag or fluorescent tag compared to methyl group can also be used as substitute. Similar types of modification that can add a fluorescent tag can also be used. In addition, any types of treatments that result in differential modification of non-methylated bases vs. methylated bases or vice versa can be used in this experiment when such a treatment leaves a detectable tag.

One such treatment is de-amination of non-methylated cytosine which may be accomplished by using sodium bisulfite. This treatment converts cystosine into uracil. The reaction itself replaces an amine group (—NH₃) with a ketone group (O═), thus as is radioactive labeling is not feasible because there is no radioactive isotope of oxygen. However, if the preexisting NH₃ is labeled with tritium in place of hydrogen then such reaction will be sufficient. Or the cytosine can first be de-aminated with sodium bisulfite and then reaminated with labeled NH₃ donor groups.

Other de-amination reactions can be modified to replace the NH₃ group with a detectable tag such as a biotin tag or a fluorescent tag. The main advantage for this method is the elimination of radioactive material usage plus fluorescent labeling allows higher density DNA array to be used. The obvious result is that more genes can be examined with the same amount of sample. Certain disadvantage that should be considered is that the DNA samples may not be comparable due to labeling agents interfering with enzymatic digestion resulting in differential digestion. One way to overcome the problem is to carry out the restriction enzyme digestion before the differentially modification (labeling) reaction using methylation insensitive enzymes. As a result, predigested DNA can be modified significantly and still binds to complementary sequences further improves the versatility of this method.

A kit containing most of the reagents described above can be used conveniently for DNA methylation studies. Such a kit should contain the appropriate reagents such as DNA methylase and methyl donor or other methylating agents, radioactive S-Adenosyl-Methionine with ³H or ¹⁴C labeled donor methyl groups or both. The kit can also contain DNA arrays needed to perform the experiment and the appropriate restriction enzymes and buffers. Such a kit can be made and sold for convenient DNA methylation studies.

The array used normally comprises aptamers made of DNA, but other type of nucleic acids such as RNA can also be used. The DNA can be from natural sources or synthetic sources. While single stranded format is preferred, double-stranded DNA can also work at reduce efficiency. Single-stranded DNA can be made with synthetic oligo nucleotides or by phage-produced single-stranded DNA.

The above description provides a methodology for which a broad spectrum of particular analysis may apply. Accordingly, the following examples are provided to further enhance the users understanding of the invention but in no way are intended to limit the particular application of the method described above.

### DESCRIPTION OF THE PREFERRED EMBODIMENTS

Single Labeling

Label a DNA sample using a reaction that can differentially modify non-methylated DNA bases vs. methylated DNA bases with a detectable label. One such reaction is methylation itself. For easy detection and quantification, methyl groups can have radioactive isotopes such as ³H or ¹⁴C. First, methylating DNA externally with radioactive methyl groups and digesting them into smaller fragments. Then, profiling these fragments on a DNA array designed with just enough aptamers so that every spots will be substantially saturated. Radiation from each spot is quantified and used to determine the amount of newly added methyl groups. From this amount, the amount of preexisting methylation is determined. Additionally, this amount can be compared to an amount on another DNA array used in a similar assay with another sample.

To control for methylation efficiency between assays, fixed amount of exogenous DNA with different degree of methylation can be added to each DNA sample prior to the methylation reaction as a calibration standard. Aptamers for these DNA standards must be present on the DNA array used. Synthetic DNA can easily be made with any modified bases desirable. Calibration standards are made to represent the percentage of DNA methylation within the range most frequently detected. This range is determined empirically and varies from array to array.

Dual Labeling

DNA methylation from two samples of DNA can be compared by externally methylating one sample with ³H-methyl groups and methylating the other sample with ¹⁴C-methyl groups. After allowing the methylation reaction to proceed to completion, unreacted methylating agents are removed and both DNA samples are mixed together for digestion by restriction enzyme. Then the resulting DNA fragments are bound to a DNA array to profile DNA fragments from both samples. Identical DNA fragments will compete for the same binding sites containing their complementary sequence. After binding and washing, radiation signals from each spot on the array are detected and quantified. Additionally, radiation signals from tritium are quantified separately from radiation signals from carbon-14. The two signals are compared to identify differentially methylated genes.

To speed up the quantitative process, the same type of exogenous DNA is added to each sample prior to treatments so that there are standard references for different degree of methylation. In addition, the same exogenous DNA captured at the same spot also has the same degree of methylation thus yield reference signal ratio for ³H/¹⁴C representative of equal methylation in both samples. To make differential quantification and quantitative comparison of signals from ³H and ¹⁴C more reliable, an array having spots of different percentage mixture of ³H and ¹⁴C can be used as reference. Advanced in computer and software allow readings of such reference standards to be stored in a computer for use in making future calculations.

Hybridization Method

Common DNA array methods are still considered qualitative and not quantitative enough for use reliably with these DNA methylation studies. There is an inherent problem when hybridizing double stranded DNA fragments to a DNA array: the DNA can rehybridize with its original complementary sequence instead of those on the array. As a result, most DNA array analysis results are considered qualitative, where only significant differences are believable. To enable accurate quantitative analysis, better hybridization method is required.

Our method of hybridization uses a temperature gradient to maximize hybridization efficiency. DNA arrays are constructed with ssDNA, typically oligos of approximately 75 bases are found to be most efficient. Furthermore, sequences with approximately the same GC percentage are chosen so that each DNA array can have a preferred hybridization temperature. For analysis, DNA array is placed in a temperature control hybridization chamber so that temperature can be raised for denaturation and then lower for hybridization. During hybridization, the temperature of the DNA array is kept lower than the temperature of the hybridization solution away from the array. In effect, a temperature gradient is created so that temperature is the lowest at the array and increase with distance away from the array.

During hybridization, dsDNA molecules that can move away from the array are exposed to higher temperature and denatured into ssDNA. These ssDNA then can move toward the array where some will hybridize with ssDNA aptamers on the array, and some will rehybridize with loose ssDNA (from the original dsDNA). Those that hybridized to aptamers are captive and will remain there because the temperature is not sufficient to denature them. Those that hybridized to another loose ssDNA can migrate back to the hotter side of the hybridization chamber where they can be denatured so they can start the whole process over again. The process will continue until all aptamers on the DNA array are saturated or there is no more DNA left to capture. Overall, this type of temperature gradient favors more DNA hybridization to the array thus achieves higher efficiency. In addition, competing loose DNA is no longer an issue, thus the result is now quantitative.

DNA Array

Single-stranded DNA array is preferred, but ssRNA array can also be used. Double-stranded DNA array does not yield good hybridization, even when used with the temperature gradient hybridization method. Most DNA array can be made by synthetic nucleotides, thus creating ssDNA is fairly simple and is preferable. Other sources of ssDNA are phage expression vectors. An ideal array for hybridization is one made with ssDNA but having both complementary sequences not hybridizing together. Synthetic ssDNA can have one sequence at one spot and its complementary sequence at a different spot. Because DNA methylation patterns are not identical on both strands of the DNA under analysis, results from each spot serve its own purpose.

One way a ssDNA array can be made from a dsDNA source is by making dsDNA with one methylated strand. DNA from a PCR reaction can be amplified for an additional cycle using methylated nucleotides. Premethylated DNA from bacteria can be amplified one cycle using regular (non-methylated) nucleotides. After immobilization to an array and preferably just right before use, the array is treated with an enzyme that differentially digest methylated DNA vs. non-methylated DNA. The resulting array has aptamers comprising of both complementary sequences of approximately equal proportion. These types of array make hybridization much more efficient because there will be no excess amount of one strand of DNA left over at the end.

Labeling Reagents

The labeling reagents of choice are enzymes and substrates pair such as DNA methylase and S-Adenosyl-Methionine, or a single DNA methylation agent that can gently methylate DNA. Enzymes can selectively label cytosines on CpGs while chemical methylating agent can methylate all non-methylated bases. Either reagent should be able to produce quantifiable results that can be used to calculate preexisting amount of methylation.

### EXAMPLES

### Example 1

### Comparing the Degree of DNA Methylation Between Two Sources of DNA

DNA from two populations of cells is compared for the purpose of discovering any difference in methylation of certain genes that can later be used as biomarkers. For instance, two set of DNA isolated from B cells from a lymphoma patient at different stage of disease progression or treatment can be compared. Equal amount of exogenous DNA is added to each sample. This DNA is synthesized to contain various percentages of methylated bases and non-methylated sites. The DNA samples are treated separately with methyl transferase using ³H labeled methyl groups for one set and ¹⁴C labeled methyl groups for the other set from the universal methyl donor S-Adenosyl-Methionine. Certain empty methylation sites on both DNA are methylated with labeled methyl groups. The DNA are cleaned from methylating reagent and mixed together. The mixture is subjected to restriction enzyme digestion and then profiled on a DNA array. Signal from ³H and ¹⁴C are differentially detected by first exposing directly to a phosphor-imaging screen and then exposing to the same screen but with a thin blocking screen for the same amount of time. Spots capturing exogenous DNA are used as reference standard to determine relative isotope ratio representing equal amount of methylation in both samples. These spots can also control for methylation efficiency. The best way to determine isotopes' quantity and ratio is to have an array of these mixtures at different quantity and ratio quantified and stored by the same instrument as reference scale for calculation.

### Example 2

### DNA Methylation Profiling for Longitudinal Comparison

DNA extracted from a cell type from normal donors is used to set a standard for future clinical assays. A fixed amount of purified DNA is used with a standardized procedure where treatments are done with precisely the same amount of reagents and treatment time. This amount of DNA is calculated to be sufficient for saturating all the aptamers on the DNA array that will be used for DNA profiling later on. A fixed amount of exogenous DNA is added to this sample prior to analysis. Then the sample is methylated chemically or enzymatically with labeled methyl groups. Finally, these DNA are digested with restriction enzyme and then profiled on a DNA array. Signals read from this array are stored for later comparison with signals read from other arrays resulting from DNA samples undergoing similar analysis.

The results are more comparable across different arrays when the amount of DNA used is sufficient to saturate all aptamer spots on every array used. One way to determine the amount of DNA sufficient for saturation is by testing different amount until any further increase in DNA won't increase the signal read. Such a study can also be performed with DNA labeled by other means such as with fluorescent dyes and radioactive nucleotides.

I claim:

1. A method of analysis comprising the steps of:
(a) treating DNA with a modifying agent that differentially modifies non-methylated DNA base versus methylated DNA base with a label; and
(b) profiling said DNA on an array.

2. The method of claim 1 further comprising a step of digesting said DNA with a restriction enzyme.

3. The method of claim 1 or claim 2 wherein a temperature gradient is used for profiling said DNA on said array.

4. The method of analysis of claim 1-3 further comprising a step of reading and comparing labels from at least two arrays.

5. The method of claim 1 wherein said modifying agent add a methyl group to certain non-methylated DNA bases.

6. The method of claim 1 further comprising a step of adding a known amount of exogenous DNA to said DNA prior to analysis.

7. The method of claim 1 wherein said modifying agent leaves a quantifiable tag on the DNA base that it modifies.

8. A method analysis comprising the steps of:
(a) methylating DNA with a labeled methyl donor;
(b) digesting said DNA with a restriction enzyme; and
(c) profiling said DNA on an array.

9. The method of claim 8 wherein said labeled methyl donor is radioactively labeled

10. The method of claim 8 wherein said labeled methyl donor is a chemical methylating agent.

11. The method of claim 8 wherein said labeled methyl donor is a substrate for a chemical or enzymatic reaction.

12. The method of claim 8 applied to two samples of DNA for methylation comparison.

13. The method of claim 8 wherein methylation reaction is done by an enzyme.

14. The method of claim 8 wherein methylation reaction is done chemically.

15. The method of claim 8 wherein methylating reaction only methylates cytosine residues of CpG in said DNA.

16. The method of claim 8 wherein methylating reaction only methylates cytosine residues of CpNpG in said DNA.

17. A method of comparing DNA methylation in two samples comprising the steps of:
(a) methylating a first sample with methyl groups containing ³H;
(b) methylating a second sample with methyl groups containing ¹⁴C;
(c) mixing said first sample and said second sample together for digestion with a restriction enzyme; and
(d) profiling the mixture on an array.

18. The method of claim 17 further comprises a step of differentially quantifying radiation signals from tritium and carbon-14.

19. The method of claim 17 wherein a temperature gradient is used for profiling said mixture on said array.

20. The method of claim 17 further comprising a step of comparing ³H's signal and ¹⁴C's signal from each spot on said array.

21. The method of claim 17 further comprising a step of comparing ³H/¹⁴C signal ratio between spots on said array.

22. The method of claim 17 further comprising a step of adding equal amount of exogenous DNA to said first sample and said second sample prior to analysis.
